(19) United States
(12) Patent Application Publication
(10) Pub. No.: US 2014/0343401 A1
(43) Pub. Date: Nov. 20, 2014

(54) SYSTEMS AND METHODS FOR CONSIDERING TARGET MOTION IN MEDICAL FIELD

(71) Applicants: Michael Huber, Beinwil am See (CH); Jonas Honegger, Zurich (CH); Mu Young Lee, San Jose, CA (US)

(72) Inventors: Michael Huber, Beinwil am See (CH); Jonas Honegger, Zurich (CH); Mu Young Lee, San Jose, CA (US)

(21) Appl. No.: 13/893,740

(22) Filed: May 14, 2013

(51) Int. Cl. A61N 5/10; A61B 6/03; A61B 8/08; A61B 19/00; A61B 5/055

(52) U.S. Cl. CPC A61N 5/1039; A61B 19/54; A61B 5/055; A61B 8/0833; A61B 6/032; A61B 6/037; A61B 19/5225; A61B 2019/5466; A61B 2019/5454; A61B 2019/5425
USPC 600/414; 600/407; 600/426; 600/437; 600/1

(57) ABSTRACT

A medical method includes: obtaining marker positions at a plurality of time points; determining a first subset of the marker positions that belongs to a first phase bin; using the marker positions in the first subset to determine a first variance information, wherein the first variance information is determined using a processor; and storing the first variance information in a non-transitory medium.

### FIELD

This application relates to systems and methods for determining information regarding tissue position, and to systems and methods that use such information regarding tissue position for treatment planning and treatment delivery purposes.

### BACKGROUND

Radiation therapy, also known as radiotherapy, has been employed to treat tumorous tissue. Radiation therapy has also been used to treat non-tumor lesions, such as malformed regions of blood vessels (ateriovenous malformation), for pain relief, to kill white blood cells before a bone marrow transplant, etc. In radiation therapy, a high energy beam is applied from an external source towards the patient. The external source, which may be rotating (as in the case for arc therapy), produces a collimated beam of radiation that is directed into the patient to the target site. The dose and placement of the dose must be accurately controlled to ensure that the tumor receives sufficient radiation, and that damage to the surrounding healthy tissue is minimized.

Sometimes during a radiotherapy, the patient may be undergoing breathing motion. In such cases, it may be desirable to monitor the breathing motion of the patient during the treatment delivery session such that radiation may be properly delivered, or ceased to be delivered, to the target region. For example, if the patient's breathing becomes non-periodic (e.g., due to sudden movement such as coughing), then it may be desirable to stop a delivery of radiation.

For accurate planning of the radiotherapy treatment, the assessment of the tumor position and its motion is very important. Especially in body parts that are subject to respiratory motion such as, in the liver or lung, the position of a tumor may vary heavily within one respiratory cycle or between different respiratory cycles. In today's treatment planning, 4D CT imaging is used to access the anatomy and tumor position in 3D for n different phases of the respiratory cycle. Due to the time-resolved acquisition process of such a 4D CT, each of the n reconstructed 3D data sets is an averaged representation of the patient's anatomy for a specific respiratory phase. The information on the variability of the anatomy for the same phase of different respiratory cycles is unaccounted for.

Applicant of the subject application has determined that it would be desirable to provide a new device and method for determining information on the variability of the anatomy for the same phase of different respiratory cycles. Applicant of the subject application has also determined that it would also be desirable to provide a new device and method for determining a treatment plan using such determined information.

### SUMMARY

A medical method includes: obtaining marker positions at a plurality of respective time points; determining a first subset of the marker positions that belongs to a first phase bin; using the marker positions in the first subset to determine a first variance information, wherein the first variance information is determined using a processor; and storing the first variance information in a non-transitory medium.

Optionally, the obtained marker positions comprises positions of a marker inside a patient.

Optionally, the obtained marker positions comprises positions of a marker outside a patient.

Optionally, the method further includes: determining a second subset of the marker positions that belongs to a second phase bin; and using the marker positions in the second subset to determine a second variance information.

Optionally, the first variance information comprises a probability distribution of the marker positions in the first set.

Optionally, the method further includes transforming the probability distribution in a marker space to a tissue space.

Optionally, the transformed probability distribution in the tissue space represents uncertainty of a position of a tissue due to motion, and the method further comprises displaying a graphic representing the uncertainty of the position of the tissue.

Optionally, the graphic comprises a plurality of lines or a color map surrounding an image or a contour of the tissue.

Optionally, the first variance information comprises a probability distribution of the marker positions in the first set, and the method further comprises displaying a graphic in a screen that is associated with the probability distribution of the marker positions in the first set.

Optionally, the graphic comprises a plurality of lines or a color map surrounding an image or a contour of a tissue.

Optionally, the graphic comprises a plurality of lines or a color map surrounding an indicator of a marker.

Optionally, the graphic represents different probability values that are above a set threshold.

Optionally, the first variance information comprises a parameter representing a number of one or more marker positions in the first set that are at a same location or within a same spatial area.

Optionally, the first variance information is determined by: determining whether one of the marker positions is at a same location or within a same spatial area as that of another one of the marker positions; and incrementing a count number associated with the location or the spatial area if the one of the marker positions is at the same location or within the same spatial area as that of the other one of the marker positions.

Optionally, the method further includes: obtaining image data; and associating the image data with the first variance information.

Optionally, the image data comprises CT image data, x-ray image data, PET image data, MR image data, SPECT image data, PET-CT image data, or ultrasound image data.

Optionally, the marker positions are obtained during a treatment procedure.

Optionally, the treatment procedure comprises a radiation treatment procedure.

Optionally, the marker positions are obtained during a 4D imaging procedure.

Optionally, the marker positions are obtained during a data collection procedure that does not involve imaging or treatment of a patient.

Optionally, the method further includes using the first variance information to determine a first parameter in a treatment plan.

Optionally, the treatment plan comprises a radiation treatment plan.

Optionally, the first parameter comprises a first treatment margin that is determined during a treatment session.

Optionally, the method further includes: determining a second subset of the marker positions that belongs to a second phase bin; using the marker positions in the second subset to determine a second variance information; and using the second variance information to determine a second treatment margin; wherein the first margin corresponds to a first phase of a physiological, and the second margin corresponds to a second phase of the physiological.

Optionally, the first parameter comprises a gating window for activating a radiation beam.

An apparatus includes a processing unit configured for: obtaining marker positions at a plurality of respective time points, determining a first subset of the marker positions that belongs to a first phase bin, and using the marker positions in the first subset to determine a first variance information; and a non-transitory medium for storing the first variance information.

Optionally, the processing unit is further configured for: determining a second subset of the marker positions that belongs to a second phase bin; and using the marker positions in the second subset to determine a second variance information.

Optionally, the first variance information comprises a probability distribution of the marker positions in the first set.

Optionally, the processing unit is further configured for transforming the probability distribution in a marker space to a tissue space.

Optionally, the transformed probability distribution in the tissue space represents uncertainty of a position of a tissue due to motion, and the processing unit is further configured to output a graphic for display in a screen, the graphic representing the uncertainty of the position of the tissue.

Optionally, the graphic comprises a plurality of lines or a color map surrounding an image or a contour of the tissue.

Optionally, the first variance information comprises a probability distribution of the marker positions in the first set, and the processing unit is further configured to output a graphic for display in a screen, the graphic associated with the probability distribution of the marker positions in the first set.

Optionally, the graphic comprises a plurality of lines or a color map surrounding an image or a contour of a tissue.

Optionally, the graphic comprises a plurality of lines or a color map surrounding an indicator of a marker.

Optionally, the graphic represents different probability values that are above a set threshold.

Optionally, the first variance information comprises a parameter representing a number of one or more marker positions in the first set that are at a same location or within a same spatial area.

Optionally, the processing unit is configured to determine the first variance information by: determining whether one of the marker positions is at a same location or within a same spatial area as that of another one of the marker positions; and incrementing a count number associated with the location or the spatial area if the one of the marker positions is at the same location or within the same spatial area as that of the other one of the marker positions.

Optionally, the processing unit is further configured for: obtaining image data; and associating the image data with the first variance information.

Optionally, the image data comprises CT image data, x-ray image data, PET image data, MR image data, SPECT image data, PET-CT image data, or ultrasound image data.

Optionally, the processing unit is further configured to use the first variance information to determine a first parameter in a treatment plan.

Optionally, the treatment plan comprises a radiation treatment plan.

Optionally, the first parameter comprises a first treatment margin, and the processing unit is configured to determine the first treatment margin during a treatment session.

Optionally, the processing unit is further configured for: determining a second subset of the marker positions that belongs to a second phase bin; using the marker positions in the second subset to determine a second variance information; and using the second variance information to determine a second treatment margin; wherein the first margin corresponds to a first phase of a physiological, and the second margin corresponds to a second phase of the physiological.

Optionally, the first parameter comprises a gating window for activating a radiation beam.

A computer product having a non-transitory medium storing a set of instructions, and execution of which causes a method to be performed, the method includes: obtaining marker positions at a plurality of respective time points; determining a first subset of the marker positions that belongs to a first phase bin; using the marker positions in the first subset to determine a first variance information, wherein the first variance information is determined using a processor; and storing the first variance information.

Optionally, the method further comprises: determining a second subset of the marker positions that belongs to a second phase bin; and using the marker positions in the second subset to determine a second variance information.

Optionally, the first variance information comprises a probability distribution of the marker positions in the first set.

Optionally, the method further comprises transforming the probability distribution in a marker space to a tissue space.

Optionally, the transformed probability distribution in the tissue space represents uncertainty of a position of a tissue due to motion, and the method further comprises outputting a graphic for display in a screen, the graphic representing the uncertainty of the position of the tissue.

Optionally, the graphic comprises a plurality of lines or a color map surrounding an image or a contour of the tissue.

Optionally, the first variance information comprises a probability distribution of the marker positions in the first set, and the method further comprises outputting a graphic for display in a screen, the graphic associated with the probability distribution of the marker positions in the first set.

Optionally, the graphic comprises a plurality of lines or a color map surrounding an image or a contour of a tissue.

Optionally, the graphic comprises a plurality of lines or a color map surrounding an indicator of a marker.

Optionally, the graphic represents different probability values that are above a set threshold.

Optionally, the first variance information comprises a parameter representing a number of one or more marker positions in the first set that are at a same location or within a same spatial area.

Optionally, the first variance information is determined by: determining whether one of the marker positions is at a same location or within a same spatial area as that of another one of the marker positions; and incrementing a count number associated with the location or the spatial area if the one of the marker positions is at the same location or within the same spatial area as that of the other one of the marker positions.

Optionally, the method further comprises: obtaining image data; and associating the image data with the first variance information.

Optionally, the image data comprises CT image data, x-ray image data, PET image data, MR image data, SPECT image data, PET-CT image data, or ultrasound image data.

Optionally, the method further comprises using the first variance information to determine a first parameter in a treatment plan.

Optionally, the first parameter comprises a first treatment margin that is determined during a treatment session.

Optionally, the first parameter comprises a gating window for activating a radiation beam.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates a system that may be used to implement one or more embodiments described herein;

FIG. 2 illustrates a medical method performed using the apparatus of FIG. 1 in accordance with some embodiments;

FIG. 3 illustrates an example of marker positions;

FIG. 4 illustrates an example of tumor positions, and respective marker positions;

FIG. 5 illustrates an example of a graphic representing a variability of tissue positions;

FIG. 6 illustrates another example of a graphic representing a variability of tissue positions;

FIG. 7 illustrates a radiation system that may be used to implement the methods described herein;

FIG. 8 illustrates another radiation system that may be used to implement the methods described herein; and

FIG. 9 is a block diagram of a computer system architecture, with which embodiments described herein may be implemented.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not explicitly described.

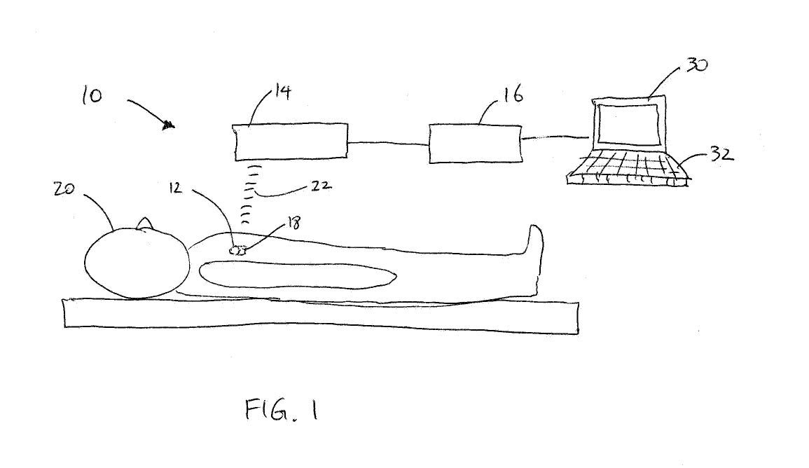

FIG. 1 illustrates an apparatus 10 in accordance with some embodiments. The apparatus 10 includes a marker 12, a sensor 14 for sensing a signal indicative of a position of the marker 12, and a processing unit 16 communicatively coupled to the sensor 14 (e.g., wirelessly or via a conductor). In the illustrated embodiments, the marker 12 comprises an implantable device having a signal transmitter 18. During use, the marker 12 is implanted inside the patient 20, and the signal transmitter 18 emits a signal 22 for detection by the sensor 14. The sensor 14 detects the signal 22 that is indicative of a position of the marker 12, and output positional data to the processing unit 16. In some embodiments, the positional data may be stored in a non-transitory medium, which may be a part of the processing unit 16, or communicatively coupled to the processing unit 16 (e.g., wirelessly or via a conductor). As shown in the figure, the apparatus 10 may optionally further include a screen 30 for displaying information to a user, and an input device 32 for allowing a user to enter input. In some embodiments, the processing unit 16, screen 30, and input 32 may be parts of a computer (e.g., laptop, desktop, etc.), or parts of a handheld device (e.g., an iPad, a tablet, a smart phone, etc.).

In some embodiments, the marker 12 may be configured to transmit radiofrequency (RF) signal, and the sensor 14 may be a receiver configured to receive the RF signal. In other embodiments, the marker 12 may be configured to emit electromagnetic field, and the sensor 14 may be a magnetic field sensor. In further embodiments, the marker 12 may be configured to emit other types of signals, and the sensor 14 may be configured to receive or sense the corresponding types of signals. In other embodiments, instead of having a transmitter 18 that actively transmits positional signals, the marker 12 may be a passive marker, which position may be detectable using the sensor 14.

The processing unit 16 may be implemented using one or more processors, such as one or more general purpose processors, one or more FPGA processors, one or more ASIC processors, or any combination of different types of processors. Also, in some embodiments, the processing unit 16 may be implemented using hardware (circuit), software, and/or combination of hardware and software.

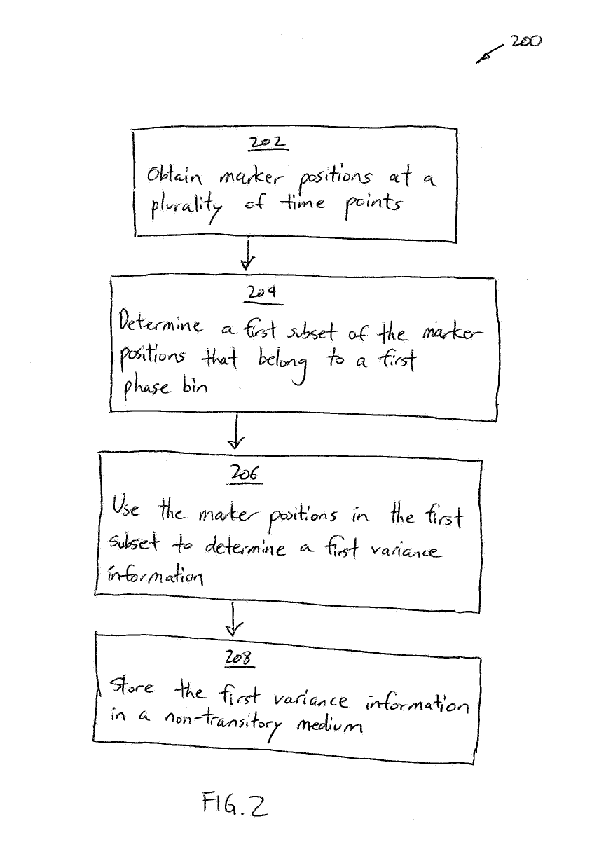

FIG. 2 illustrates a medical method 200 performed using the device 10 of FIG. 1 in accordance with some embodiments. First, marker positions at a plurality of time points are obtained (Item 202). In some embodiments, such may be accomplished using the sensor 14 to detect the marker 12 at different time points. The sensor 14 then generates signals representative of the positions of the marker 12 at the different time points. In some embodiments, the signals provided from the sensor 14 may be considered marker positions. In other embodiments, the signals from the sensor 14 may be transmitted to a processing unit 16, which calculates the marker positions using the signals from the sensor 14. Thus, the act of obtaining marker positions may be performed by the sensor 14, by the processing unit 16, or by both the sensor 14 and the processing unit 16 in different embodiments. In further embodiments, the marker positions may be stored in a non-transitory medium. In such cases, the act of obtaining the marker positions may be performed by a device (which may be the processing unit 16, or another processing unit) that retrieves the stored marker positions.

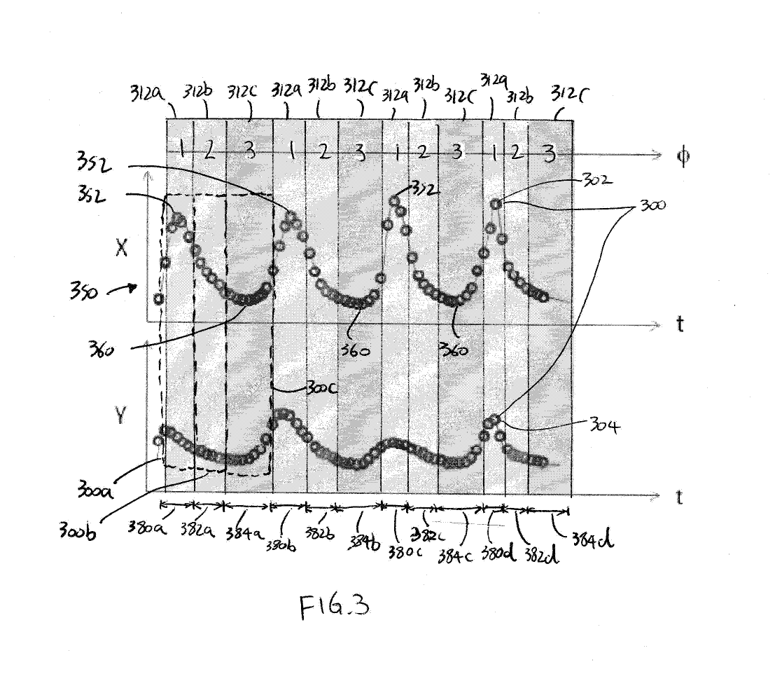

In some embodiments, each marker position may be a two-dimensional coordinate (e.g., having X, Y components). FIG. 3 illustrates an example of marker positions 300 obtained over a period of time. In the illustrated example, each marker position has a X-component 302, and a Y-component 304. In other embodiments, each marker position may have only one component, or may have three components (e.g., X, Y, Z components).

Returning to FIG. 2, next, the processing unit 16 determines a first subset of the marker positions that belongs to a first phase bin (Item 204). In some embodiments, the marker positions are obtained while the patient 20 is breathing, and the resulting marker positions correspond with the breathing motion. Thus, the marker positions may exhibit a periodic pattern, like that shown in the example of FIG. 3. In some embodiments, different marker positions may be assigned to different phases of a respiratory cycle. In the illustrated example of FIG. 3, a respiratory cycle is divided into three phase bins 312a-312c, which correspond with three respective phase ranges of the respiratory cycle. Phase bin 312a covers the first ⅓ portion of the physiological cycle, phase bin 312b covers the middle ⅓ portion of the physiological cycle, and phase bin 312c covers the last ⅓ of the physiological cycle. In some embodiments, the phase range in a physiological cycle may be divided evenly among the different phase bins. In other embodiments, the phase range in a physiological cycle may be divided un-evenly among the different phase bins. Although three phase bins 312a-312c are shown in the example, in other embodiments, there may be fewer than three phase bins 312 (e.g., two phase bins 312), or more than three phase bins 312.

As shown in FIG. 3, for a given phase bin 312, there may be multiple marker positions that are assigned to that given phase bin 312. For example, the marker positions 300a (which are a subset of all of the marker positions 300 obtained) may be assigned to phase bin 312a by the processing unit 16 during item 204. Similarly, the processing unit 16 may also assign the marker positions 300b to phase bin 312b, and the marker positions 300c to phase bin 312c, during Item 204.

FIG. 3 also illustrates a technique that may be used by the processing unit 16 to bin marker positions based on phase. As shown in the figure, from the pattern 350 of the marker positions, the processing unit 16 may identify peaks 352 in the pattern 350. Each peak 352 corresponds with a peak amplitude of a respiratory cycle, and therefore, may be associated with an end of an inhale phase (or a beginning of an exhale phase) in a respiratory cycle. Once the peaks 352 have been identified, the processing unit 16 may then determine that the marker position at the peak 352 corresponds to a beginning phase (or an end phase) of a physiological cycle. A phase of a respiratory cycle represents a degree of completeness of the respiratory cycle. In the illustrated example, a phase value of 0° (and 360°) represents a peak of an inhale state, and the phase value varies linearly between 0° and 360° in a physiological cycle. Following the above example in which the number of phase bins is three, all marker positions with corresponding phase values from 0°-120° (marker positions obtained at time periods 380a-380d) would be binned into phase bin 312a, all marker positions with corresponding phase values from 120°-240° (marker positions obtained at time periods 382a-382d) would be binned into phase bin 312b, and all marker positions with corresponding phase values from 240°-360° (marker positions obtained at time periods 384a-384d) would be binned into phase bin 312c. Note that the duration of the time periods 380a-380d for phase bin 312a in the example are not necessarily equal, and that they may be different, depending on the breathing pattern of the patient. Similar is true for the time periods for phase bin 302b and phase bin 302c.

In the above example, the peak 352 in the pattern of the marker positions is associated with a phase value that is 0° or 360°. In other embodiments, the phase value associated with the peak 352 may be different from 0° and 360°, and may be assigned an arbitrary phase value.

In other embodiments, instead of identifying the peaks 352, the processing unit 16 may be configured to identify the low points 360 in the pattern 350. Each low point 360 corresponds with a minimum amplitude of a respiratory cycle, and therefore, may be associated with an end of an exhale phase (or a beginning of an inhale phase) in a respiratory cycle. Once the low points 360 have been identified, the processing unit 16 may then determine that the marker position at the low point 360 corresponds to a beginning phase (or an end phase) of a physiological cycle. For example, a phase value of 0° (and 360°) may represent an end of an exhale state, and the phase value varies linearly between 0° and 360° in a physiological cycle. In other embodiments, the phase value associated with the low point 360 may be different from 0° and 360°, and may be assigned an arbitrary phase value.

It should be noted that the number of phase bins 312 is not limited to three in the above example. In other embodiments, the physiological cycle may be divided into a number of phase bins 312 that is less than three, or more than three. In some embodiments, the user interface (e.g., the screen 30 and the input device 32) may allow a user to selectively prescribe the number of phase bins 312 (e.g., by entering a value). In other embodiments, the number of phase bins 312 may be pre-determined and fixed. Also, in other embodiments, instead of a breathing motion, the marker positions may correspond with other types of physiological motion, such as a cardiac motion, which also exhibits a periodic pattern.

Returning to the method 200 of FIG. 2, next, the processing unit 16 uses the marker positions in the subset of the obtained marker positions to determine variance information (Item 206). In some embodiments, the variance information may be (or may represent) a probability distribution of the marker positions 300 in a phase bin 312 (which corresponds with certain phase or phase range of a physiological cycle). For example, in some embodiments, the processing unit 16 may determine a probability distribution of the marker positions 300 in each of the phase bins 312a-312c. Also, in some embodiments, the variance information may be a count number representing a number of one or more marker positions 300 in a phase bin 312 that are at a same location or within a same spatial area. In such cases, the processing unit 16 may be configured to determine the variance information by determining whether one of the marker positions 300 is at a same location or within a same spatial area as that of another one of the marker positions (that are from the same phase bin 312), and incrementing a count number associated with the location or the spatial area if the one of the marker positions 300 is at the same location or within the same spatial area as that of the other one of the marker positions 300. Using such technique, marker positions 300 from the same phase bin 312 that occur multiple times, or that are within a same spatial area, get accumulated. In some embodiments, the accumulated number (count number) may be considered an example of variance information.

In other embodiments, instead of, or in addition to, accumulating number of marker positions 300 for a given point or area, the processing unit 16 may associate the marker positions 300 in the subset determined in Item 204 with each other so that they belong to the same group (e.g., same phase bin 312). In such cases, the act of determining variance information may be considered performed by the processing unit 16 when it associates the different marker positions 300 with each other.

In further embodiments, the processing unit 16 may be configured to provide a graphic (e.g., in the form of graphical signals) for display in a screen, wherein the graphic represents the variance information. In such cases, the graphic may be considered variance information, and the act of determining variance information may be considered performed by the processing unit 16 when it provides the graphic (e.g., graphical signals).

Also, in one or more embodiments, the above technique may be repeated for other phase bin(s) 312. For example, in other embodiments, the processor may determine a second subset of the marker positions 300 that belongs to a second phase bin 312, and may use the marker positions 300 in the second subset to determine a second variance information.

Returning to the method 200 of FIG. 2, in some embodiments, the variance information may be stored in a non-transitory medium (Item 208). The stored variance information may be later retrieved for processing in some embodiments. For example, in some embodiments, the stored variance information may be used to determine one or more parameters in a treatment plan. By means of non-limiting examples, the parameter(s) may include a treatment margin, a gating window for activating a radiation beam, a gating window for de-activating a radiation beam, etc. Techniques for using the variance information to determine treatment plan parameter(s) will be described in further detail herein.

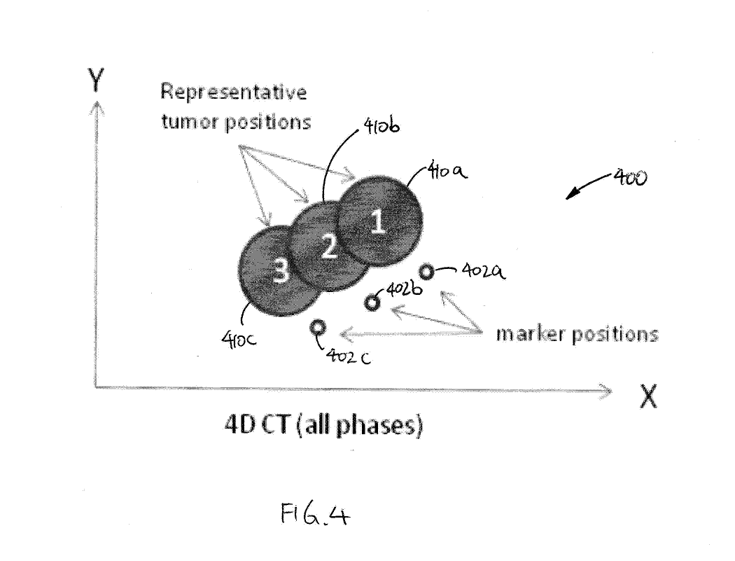

Also, in some embodiments, the variance information may be displayed in a screen for presentation to a user. As shown in FIG. 4, the average marker positions for the respective phase bins 312 may be presented in a graph 400. In the graph 400, the marker position 402a is the average of the marker positions 300 in the first phase bin 312a, the marker position 402b is the average of the marker positions 300 in the second phase bin 312b, and the marker position 402c is the average of the marker positions 300 in the third phase bin 312c. The tumor positions 410a-410c at the respective phases are also plotted to show how the marker positions 402a-402c correlate with the tumor positions 410a-410c, respectively.

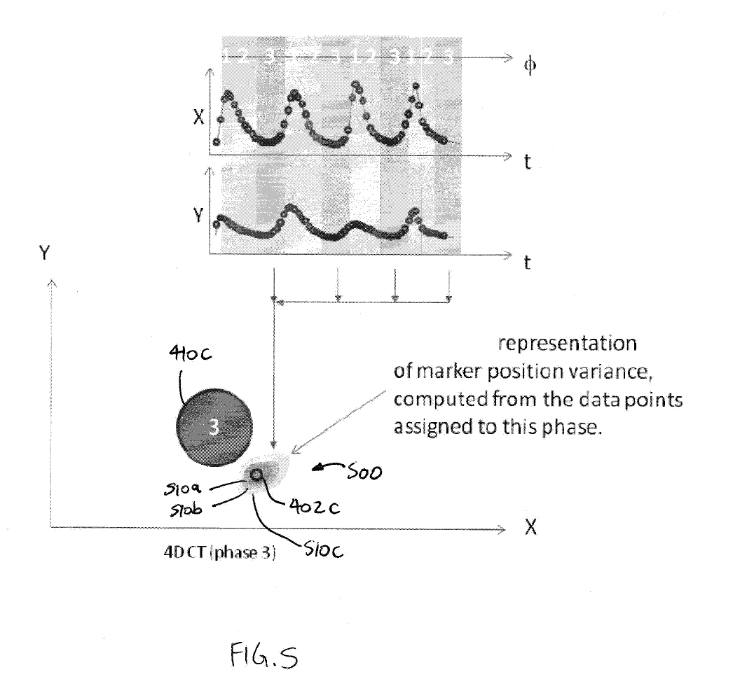

In some embodiments, the variance information may be presented in the graph 400. For example, in some embodiments, the variance information may be presented in a form of a color map 500 in the marker space like that shown in FIG. 5. Such color map 500 may be generated by the processing unit 16 and displayed in the screen 30. In the illustrated embodiments, the color map 500 includes a first region 510a with a first color representing a first probability that the marker position is in that region 510a. The color map 500 also includes a second region 510b with a second color representing a second probability that the marker position is in that region 510b, and a third region 510c with a third color representing a third probability that the marker position is in that region 510c. Although three regions 510a-510c are shown in the example, in other embodiments, there may be fewer than three regions 510, or more than three regions 510. Also, in the illustrated example, the regions 510a-510c represent respective probabilities of 90%, 95%, and 99% that the marker is in those respective regions. In other embodiments, the regions 510 may represent other probability values, respectively. In some cases, a user interface presented in the screen 30 may allow a user to selectively enter a prescribed probability threshold. In such cases, the screen 30 will display regions 510 having respective probabilities that are above the prescribed probability threshold, and not regions having probabilities that are below the prescribed probability threshold. In addition, in the illustrated example, the color map 500 is presented for one of the marker positions 402 (i.e., marker position 402c) that corresponds with one of the phase bins 312. In other embodiments, the color map 500 may be presented for another one of the marker positions 402 that corresponds with another phase bin 312.

Various techniques may be used to generate the color map 500. In some embodiments, a pixel/voxel position p(x,y,z) may be assigned a value corresponding to how many recorded marker positions are found in a surrounding circle with a defined radius R in relation to the total amount of marker positions. For example, assuming there are a total of 200 recorded marker positions 200, and radius R=1 cm. For a certain position p, there may be 150 recorded points within 1 cm of its surrounding. This means p gets a value of 150/200=0.75 (or 75%). The above calculation is repeated for each pixel/voxel in the color map 500 to generate the map 500.

In some embodiments, the map 500 may represent values of a spatial parameter p such that X % of points would be within a distance p from the average or nominal position. Because organ motion introduces some level of uncertainty for the physician when the physician defines the shape and size of the region of the body to be treated (PTV), this so-called Planning Treatment Volume may be selected by the physician to be somewhat larger than the actual tumor in order to account for the variance in tumor location. The physician may decide to define a PTV such that it encompasses X % of the range of motion of the tumor. Therefore even if the marker data is a finite number of scatter points in space as opposed to a continuous distribution, the processing unit 16 may compute a 1sigma (or 2sigma, etc.) distance in radius or a X % confidence interval to generate the map 500.

In other embodiments, instead of the color map 500, the processing unit 16 may generate other graphics for presentation in a screen that represent the variance information. For example, in other embodiments, the processing unit 16 may cause the screen to display a graphic that includes a plurality of lines (e.g., isolines) around the marker position 402.

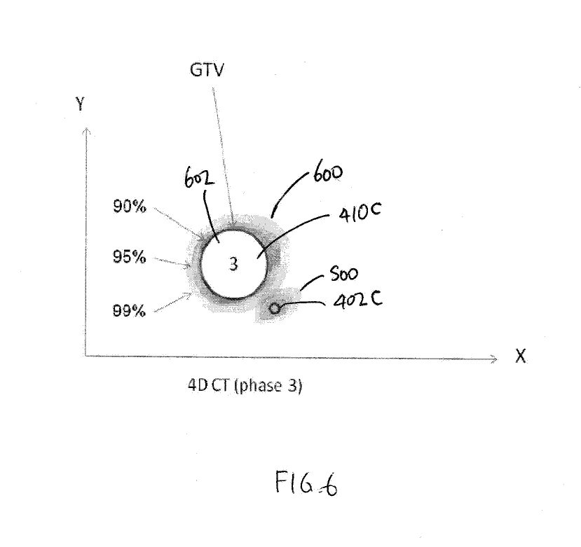

Also, in other embodiments, instead of presenting the variance information in the marker space, the processing unit 16 may present the variance information in the tissue space. For example, in other embodiments, the color map 500 representing the variance information may be presented in the tissue space like that shown in FIG. 6. Such color map 600 may be generated by the processing unit 16 and displayed in the screen 30. In one technique, the color map 500 around the marker position 402c may be mathematically “stretched” to form color map 600 and be placed around a contour of the tissue structure 602 using the processing unit 16, to thereby present the color map 600 around the tissue structure 602. In some embodiments, the color map 600 may be presented together with an image representing the tissue structure 602, which may be an actual image (e.g., x-ray image, CT image, etc.) of the tissue structure 602, or an artificially created graphic, such as a contour or a model or the tissue structure 602. In the illustrated example, the color map 600 is presented for one of the tissue positions 410 (i.e., tissue position 410c) that corresponds with the marker position 402c. In other embodiments, the color map 600 may be presented for another tissue position 410 that corresponds with another marker position 402. The color map 600 represents/indicates the probability distribution of tissue position in the tissue space, and the uncertainty of a position of a tissue due to motion.

In other embodiments, instead of the color map 600, the processing unit 16 may generate other graphics for presentation in a screen that represent the variance information in the tissue space. For example, in other embodiments, the processing unit 16 may cause the screen to display a graphic that includes a plurality of lines (e.g., isolines) around the tissue structure.

Also, in some embodiments, the color map 600 may be created by transforming the probability distribution of the marker positions in a marker space to a tissue space. In one implementation, such may be accomplished using a processor that calculates various data points in the probability distribution in the tissue space based on the marker positions and the shape of the tissue structure 602. The transformed probability distribution in the tissue space represents uncertainty of a position of a tissue due to motion.

As discussed, in some embodiments, the variance information (e.g., map 600, contour(s), count number(s), etc.) may be presented together with an image of tissue structure. In some embodiments, the image of the tissue structure may be image data. By means of non-limiting examples, the image data may be CT (e.g., CBCT) image data, x-ray image data, PET image data, MR image data, SPECT image data, PET-CT image data, ultrasound image data, kv-image data, tomosynthesis image data, etc. In other embodiments, the image of the tissue structure may be an artificially created graphic, such as a contour or a model of the tissue structure.

Also, in one or more embodiments, the marker positions and the variance information may be obtained using the device 10 during a treatment procedure. For example, in some embodiments, the marker positions may be obtained during a radiation treatment procedure, or a non-radiation treatment procedure. In other embodiments, the marker positions and the variance information may be obtained using the device 10 during an imaging procedure (e.g., during a 4D imaging procedure, such as a 4D-CT (e.g., 4D CBCT) acquisition, 4D MRI acquisition, 4D PET acquisition, 4D ultrasound acquisition, etc.). In other embodiments, the imaging procedure may be a tomosynthesis procedure, a partial tomosynthesis procedure, a MRI procedure, etc. In further embodiments, the marker positions and the variance information may be obtained during a non-imaging session and a non-treatment session.

Also, in some embodiments, there may be multiple sets of marker data acquired during different times. For example, in some embodiments, there may be a set of marker data obtained during an imaging session (e.g., during a 4D imaging procedure, such as a 4D CT (e.g., 4D CBCT) acquisition, 4D MRI acquisition, 4D PET acquisition, 4D ultrasound acquisition, etc., or during any of other types of imaging procedure, such as tomosynthesis procedure, partial tomosynthesis procedure, a MRI procedure, etc.), another set of marker data obtained during a treatment session, and another set of marker data obtained during a non-imaging and non-treatment session (e.g., outside an imaging room and a treatment room). These different sets of marker data may be obtained using the same device 10, or different devices. In some embodiments, different set of marker data may be processed to obtain different respective sets of variance information, and the different sets of variance information may be combined with different image data obtained using different imaging techniques. For example, a first variance information obtained using the first set of marker data may be combined with any one of the types of image data mentioned above, and a second variance information obtained using the second set of marker data may be combined with another one of the types of image data mentioned above. Also, in some embodiments, a map may be determined that takes multiple motion information sources into account. In some cases, using marker data obtained from different sessions to obtain variance information may allow for investigations of long term variances of a tumor motion.

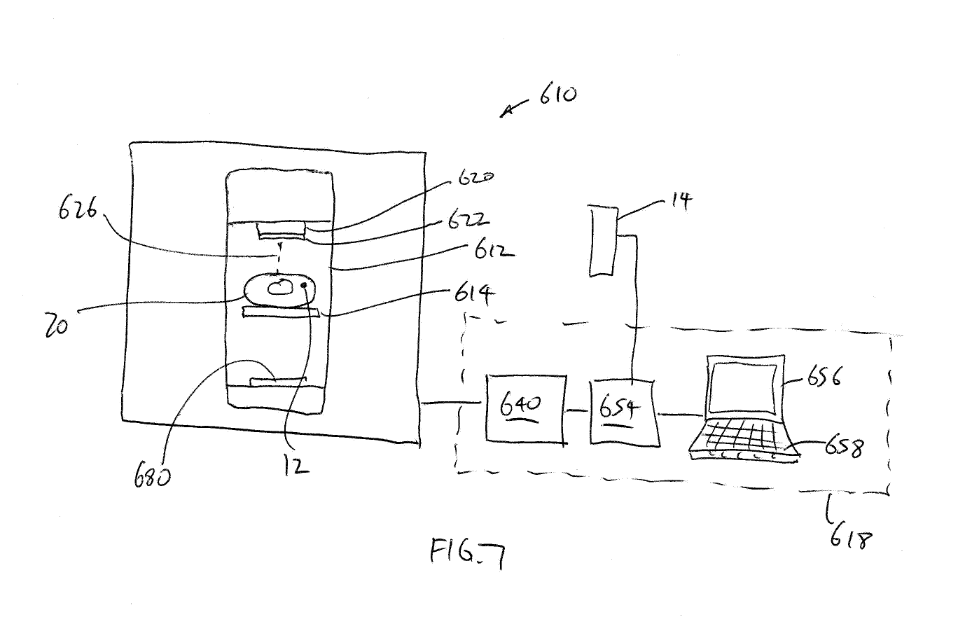

As discussed, the image of tissue structure for display with the variance information may be obtained during a treatment session. FIG. 7 illustrates a radiation treatment system 610 that may be used to obtain the image of the tissue structure. In some cases, the system 610 may be used with the device 10, or may be used to implement one or more components of the device 10, of FIG. 1. The system 610 includes an arm gantry 612, a patient support 614 for supporting a patient 20, and a control system 618 for controlling an operation of the gantry 612. The system 610 also includes a radiation source 620 that projects a beam 626 of radiation towards the patient 20 while the patient 20 is supported on support 614, and a collimator system 622 for controlling a delivery of the radiation beam 626. The radiation source 620 can be configured to generate a cone beam, a fan beam, or other types of radiation beams in different embodiments.

In the illustrated embodiments, the radiation source 620 is a treatment radiation source for providing treatment energy. In other embodiments, in addition to being a treatment radiation source, the radiation source 620 can also be a diagnostic radiation source for providing diagnostic energy for imaging purpose. In such cases, the system 610 will include an imager, such as the imager 680, located at an operative position relative to the source 620 (e.g., under the support 614). In further embodiments, the radiation source 620 may be a treatment radiation source for providing treatment energy, wherein the treatment energy may be used to obtain images. In such cases, in order to obtain imaging using treatment energies, the imager 680 is configured to generate images in response to radiation having treatment energies (e.g., MV imager). In some embodiments, the treatment energy is generally those energies of 160 kilo-electron-volts (keV) or greater, and more typically 1 mega-electron-volts (MeV) or greater, and diagnostic energy is generally those energies below the high energy range, and more typically below 160 keV. In other embodiments, the treatment energy and the diagnostic energy can have other energy levels, and refer to energies that are used for treatment and diagnostic purposes, respectively. In some embodiments, the radiation source 620 is able to generate X-ray radiation at a plurality of photon energy levels within a range anywhere between approximately 10 keV and approximately 20 MeV. In further embodiments, the radiation source 620 can be a diagnostic radiation source. In the illustrated embodiments, the radiation source 620 is carried by the arm gantry 612. Alternatively, the radiation source 620 may be located within a bore (e.g., coupled to a ring gantry).

In the illustrated embodiments, the control system 618 includes a processing unit 654, such as a processor, coupled to a control 640. The control system 618 may also include a monitor 656 for displaying data and an input device 658, such as a keyboard or a mouse, for inputting data. The operation of the radiation source 620 and the gantry 612 are controlled by the control 640, which provides power and timing signals to the radiation source 620, and controls a rotational speed and position of the gantry 612, based on signals received from the processing unit 654. Although the control 640 is shown as a separate component from the gantry 612 and the processing unit 654, in alternative embodiments, the control 640 can be a part of the gantry 612 or the processor 654. The processing unit 654 may be the processing unit 16 of the device 10 of FIG. 1, or a separate processing unit that is different from the processing units 16. Also, in some embodiments, the screen 656 may be the screen 30 of the device 10 of FIG. 1, and the input device 658 may be the input device 32 of FIG. 1. In other embodiments, the screen 656 and the input device 658 may be different from the screen 30 and the input device 32 of FIG. 1.

In some embodiments, before a treatment session performed by the system 610, or during a treatment session performed by the system 610, the device 10 may be used to perform the method 200 of FIG. 2 to thereby obtain variance information regarding a position of a marker and/or a tissue structure in the patient 20. The obtained variance information may be used to verify treatment margins prescribed in a treatment plan before the system 610 delivers radiation to treat the patient 20 according to the treatment plan. In other embodiments, the variance information may be used to adjust one or more parameters in the treatment plan.

Also, in some embodiments, one or more images obtained using the imager 680 of the system 610 may be processed to obtain an image of a tissue structure. Such image of the tissue structure may then be combined with the variance information obtained using the device 10, and be presented in the screen (like that shown in the example of FIG. 6).

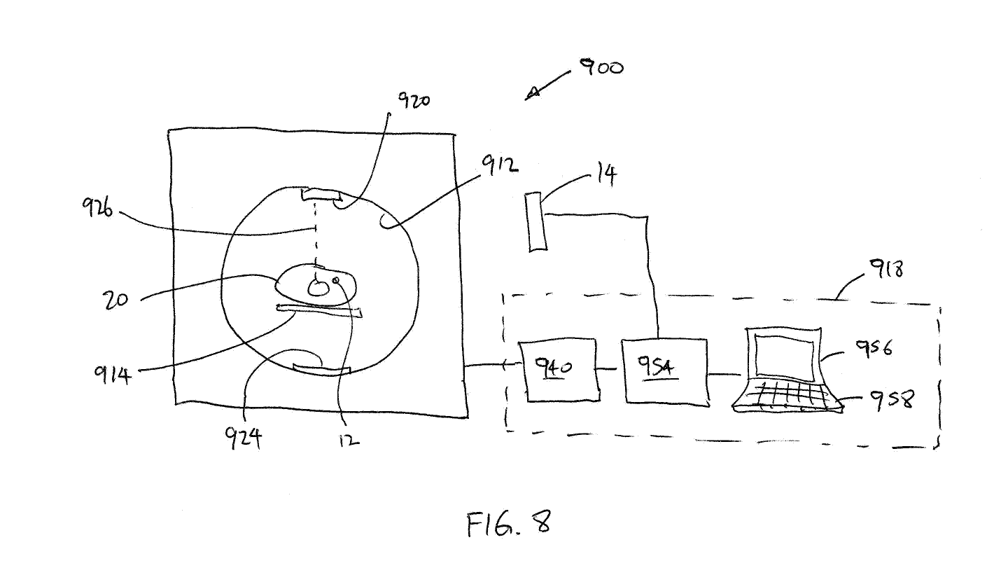

Also, as discussed, in some embodiments, the image of the tissue structure for display with the variance information may be obtained during an imaging session. FIG. 8 illustrates CT system 900 that may be used to obtain an image of tissue structure. The system 900 may be used with the device 10, or may be used to implement one or more components of the device 10, of FIG. 1 in accordance with some embodiments. The system 900 includes a gantry 912, and a support 914 for supporting a patient 20. The gantry 912 includes an x-ray source 920 that projects a beam 926 of x-rays towards a detector 924 on an opposite side of the gantry 912 while the patient 20 is positioned at least partially between the x-ray source 920 and the detector 924. By means of non-limiting examples, the beam of x-rays can be a cone beam or a fan beam. The detector 924 has a plurality of sensor elements configured for sensing a x-ray that passes through the patient 20. Each sensor element generates an electrical signal representative of an intensity of the x-ray beam as it passes through the patient 20.

The system 900 also includes a control system 918. In the illustrated embodiments, the control system 918 includes a processing unit 954, such as a computer processor, coupled to a control 940. The control system 918 may also include a monitor 956 for displaying data and an input device 958, such as a keyboard or a mouse, for inputting data. The operation of the radiation source 920 and the gantry 912 are controlled by the control 940, which provides power and timing signals to the radiation source 920, and controls a rotational speed and position of the gantry 912, based on signals received from the processing unit 954. Although the control 940 is shown as a separate component from the gantry 912 and the processing unit 954, in alternative embodiments, the control 940 can be a part of the gantry 912 or the processing unit 954. The processing unit 954 may be the processing unit 16, or a separate processing unit that is different from the processing units 16. Also, in some embodiments, the screen 956 may be the screen 30, and the input device 958 may be the input device 32 of FIG. 1. In other embodiments, the screen 956 and the input device 958 may be different from the screen 30 and the input device 32 of FIG. 1.

It should be noted that the system 900 is not limited to the configuration described above, and that the system 900 may have other configurations in other embodiments. For example, in other embodiments, the system 910 may have a different shape. In other embodiments, the radiation source 920 of the system 900 may have different ranges of motions and/or degrees of freedom. For example, in other embodiments, the radiation source 920 may be rotatable about the patient 20 completely through a 360° range, or partially through a range that is less than 360°. Also, in other embodiments, the radiation source 920 is translatable relative to the patient 20. Further, the radiation source 920 is not limited to delivering diagnostic energy in the form of x-ray, and may deliver treatment energy for treating a patient.

During a scan to acquire x-ray projection data (i.e., CT image data), the gantry 912 rotates about the patient 20 at different gantry angles, so that the radiation source 920 and the imager 924 may be used to obtain images at different gantry angles. As the system 900 is operated to obtain images at different gantry angles, the patient 20 is breathing. Thus, the resulting images at different gantry angles may correspond to different phases of a breathing cycle for the patient 20. After the scan is completed, the projection images at different gantry angles are stored, e.g., in a memory (such as a non-transitory medium), and the projection images are processed to sort the images so that images at different gantry angles that correspond to a same phase of a breathing cycle are binned (e.g., associated with each other). The binned images for a specific phase of a respiratory cycle can then be used to generate a reconstructed three-dimensional CT image for that phase. In some embodiments, the CT image (or a 2D section of such CT image) for a particular phase may be presented together with the variance information obtained using the device (like that shown in the example of FIG. 6).

In further embodiments, the marker positions may be obtained during a data collection procedure that does not involve imaging or treatment. For example, in some embodiments, the device 10 of FIG. 1 may be used in a non-imaging or non-treatment process to collect data regarding a patient's physiological motion, and to determine variance information.

In the above embodiments, the device 10 has been described with reference to an internal marker that is implanted inside a patient. In other embodiments, the marker may be located outside the patient. For example, in other embodiments, the marker may be located at a marker block that is coupled to (e.g., placed on top of) the patient. In further embodiments, the marker may be attached to a patient's skin. In still further embodiments, the marker may be implemented using a part (e.g., a landmark on a skin, an internal tissue structure, etc.) of the patient. In further embodiments, the marker may be an internal tissue (e.g., a patient's diaphragm, a surface of an organ, etc.), which may be monitored using any imaging technique, such as ultrasound, x-ray, etc. Thus, as used in this specification, the term “marker” should not be limited to a device, and may refer to any object (such as tissue).

Also, in other embodiments, the device 10 may not include the sensor 14. For example, in other embodiments, the device 10 may instead include a camera for viewing a maker (or markers) that is on or is coupled to the patient.

The marker may be a marker on a marker block placed on the patient, a fiducial secured to a patient's skin, an anatomical feature on the patient's skin, etc. The camera may be used to capture the marker movement while the patient is undergoing a physiological motion (e.g., breathing motion). The camera may transmit the images to a processing unit, which processes the images to determine marker positions at different phases of the breathing motion. The processing unit may also determine variance information for different phases or phase ranges of the breathing motion, like that described previously.

In further embodiments, the device 10 may be any device for providing positional data. For example, in other embodiments, the device 10 may be a haptic vest worn by a patient, or a strain gauge coupled to the patient, for measuring a degree of movement undergone by the patient as the patient is going through physiological motion. In still further embodiments, the device for providing positional data may be an imaging device, such as a CT machine, a x-ray, a tomosynthesis device, a PET machine, a SPECT machine, a MRI machine, an ultrasound device, etc.

In other embodiments, the variance information may be obtained by combining data sets from different sources. For example, in some embodiments, motion data from the system of FIG. 2 may be combined with imaging data from an imaging device, and the combined data may be processed by a processing unit to calculate the variance information.

In some embodiments, the variance information may be used by the processing unit 16 to determine a treatment plan. For example, in some embodiments, the processing unit 16 may be configured to use the variance information to determine one or more parameters for a treatment plan. In some embodiments, the treatment parameter may be a treatment margin for a particular phase of a physiological cycle. For example, in some embodiments, the processing unit 16 may be configured to determine different treatment margins for the different respective phases of a physiological cycle based on the variance information for the different phases. Using the techniques described herein, it is possible to determine different treatment margins for different phases of a physiological cycle. This is because different variance information may be determined by the processing unit 16 for different phase bins. For example, the processing unit 16 may determine that there is a 90% probability that a tumor varies in position by 2 cm or less at a respiratory phase=36°, and that there is a 90% probability that the tumor varies in position by 1 cm or less at a respiratory phase=88°. In such cases, the processing unit 16 may determine that the treatment margin for phase=36° is 2 cm, and that the treatment margin for phase=88° is 1 cm. In other embodiments, the treatment margins for phases=36°, 88° may be determined as 2 cm times a factor of safety, and 1 cm times a factor of safety, respectively. In further embodiments, the treatment margins for phases=36°, 88° may be determined as 2 cm plus a value, and 1 cm plus a value, respectively.

Also, in some embodiments, the amount of allowable variation between an irradiated location and a target location may depend on where the target region is with respect to the rest of the target volume. For example, if the trajectory is to treat near a border of a volume, then the margin determined based on the variance information may be narrowed or reduced to reduce the risk of irradiating healthy tissue. On the other hand, if the trajectory is to treat a target region near a center of a volume, then the amount of variation may be allowed to have a maximum value (i.e., the delivered radiation may be allowed to miss the intended position by a higher distance). This is because if the radiation misses the target region that is near a center of a target volume, the radiation may still be within the target volume, and the healthy tissue may not be at risk.

Also, in some embodiments, the treatment margin(s) may be determined using the variance information during a treatment planning that occurs before a treatment session. In other embodiments, the treatment margin(s) may be determined using the variance information during a treatment session (e.g., between deliveries of radiation beam, and while the radiation treatment machine is “ON”).

In other embodiments, the treatment parameter may be one or more gating windows for activating and/or deactivating a radiation beam. For example, in some embodiments, the processing unit 16 (or a user) may determine from the variance information that there is too much uncertainty in the position of the marker (and therefore, the position of the tumor) at a given phase of a physiological cycle (e.g., the uncertainty is above a prescribed threshold). In such cases, the processing unit 16 (or the user) may determine that the radiation beam should be turned off at that given phase. Alternatively, or additionally, the processing unit 16 (or a user) may determine from the variance information that the uncertainty in the position of the marker (and therefore, the position of the tumor) at a given phase of a physiological cycle is relatively low (e.g., the uncertainty is below a prescribed threshold). In such cases, the processing unit 16 (or the user) may determine that the radiation beam may be turned on at that given phase. Also, in some embodiments, the variance information may be used by the processing unit 16 to determine whether to perform gating at certain phase(s) of a physiological cycle during a treatment procedure. In other embodiments, the variance information may be used by the processing unit 16 to determine whether to perform tracking at certain phase(s) of a physiological cycle during a treatment procedure.

Also, in some embodiments, the variance information may be used to determine an uncertainty distribution of positions associated with a tissue structure, wherein such uncertain distribution may be represented by a Probability Density Function (PDF). Furthermore, in some embodiments, marker data may be used together with the variance information to determine one or more of a Planning Target Volume (PTV), an Internal Target Volume (ITV), a Planning Margin (PM) an Internal Margin (IM), and a Planning Organ at Risk Volume (PRV). These parameters have been described in the International Commission on Radiation Units & Measurements (ICRU), Report 62.

In some embodiments, the act of determining the treatment parameter(s) using the variance information may be performed in a treatment planning procedure before a treatment session. In other embodiments, the act of determining the treatment parameter(s) using the variance information may be performed during a treatment session, such as, before a radiation beam is activated, or between activations of radiation beams (and while the radiation treatment machine is “ON”).

It should be noted that the treatment plan is not limited to a radiation treatment plan, and may be any treatment plan that may or may not include radiation. For example, in other embodiments, the treatment parameter(s) determined using the variance information may be for a proton treatment plan.

Computer System Architecture

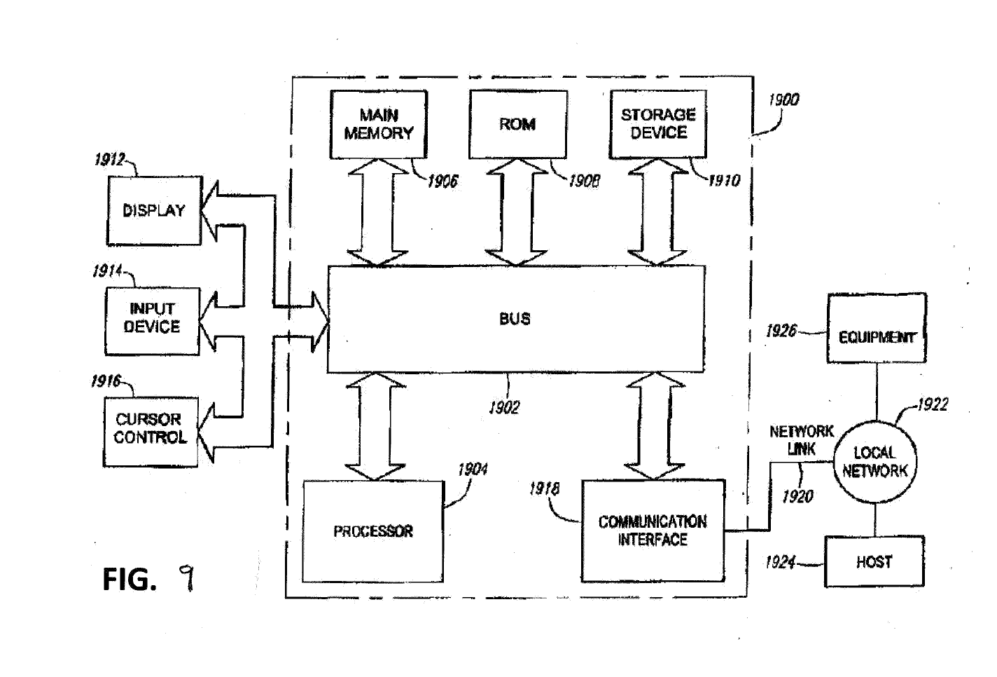

FIG. 9 is a block diagram that illustrates an embodiment of a computer system 1900 upon which an embodiment of the invention may be implemented. Computer system 1900 includes a bus 1902 or other communication mechanism for communicating information, and a processor 1904 coupled with the bus 1902 for processing information. The processor 1904 may be an example of the processing unit 16 of FIG. 1, or another processor that is used to perform various functions described herein. For example, in some embodiments, the processor 1904 may be configured to perform one or more items described with reference to the method 200 of FIG. 2.

Returning to FIG. 9, the computer system 1900 also includes a main memory 1906, such as a random access memory (RAM) or other dynamic storage device, coupled to the bus 1902 for storing information and instructions to be executed by the processor 1904. The main memory 1906 also may be used for storing temporary variables or other intermediate information during execution of instructions to be executed by the processor 1904. The computer system 1900 further includes a read only memory (ROM) 1908 or other static storage device coupled to the bus 1902 for storing static information and instructions for the processor 1904. A data storage device 1910, such as a magnetic disk or optical disk, is provided and coupled to the bus 1902 for storing information and instructions.

The computer system 1900 may be coupled via the bus 1902 to a display 1912, such as a cathode ray tube (CRT) or a flat panel, for displaying information to a user. An input device 1914, including alphanumeric and other keys, is coupled to the bus 1902 for communicating information and command selections to processor 1904. Another type of user input device is cursor control 1916, such as a mouse, a trackball, or cursor direction keys for communicating direction information and command selections to processor 1904 and for controlling cursor movement on display 1912. This input device typically has two degrees of freedom in two axes, a first axis (e.g., x) and a second axis (e.g., y), that allows the device to specify positions in a plane.

The computer system 1900 may be used for performing various functions (e.g., calculation) in accordance with the embodiments described herein. According to one embodiment, such use is provided by computer system 1900 in response to processor 1904 executing one or more sequences of one or more instructions contained in the main memory 1906. Such instructions may be read into the main memory 1906 from another computer-readable medium, such as storage device 1910. Execution of the sequences of instructions contained in the main memory 1906 causes the processor 1904 to perform the process steps described herein. One or more processors in a multi-processing arrangement may also be employed to execute the sequences of instructions contained in the main memory 1906. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to the processor 1904 for execution. Such a medium may take many forms, including but not limited to, non-volatile media, volatile media, and transmission media. Non-volatile media includes, for example, optical or magnetic disks, such as the storage device 1910. A non-volatile medium may be considered as an example of a non-transitory medium. Volatile media includes dynamic memory, such as the main memory 1906. A volatile medium may be considered as another example of a non-transitory medium. Transmission media includes coaxial cables, copper wire and fiber optics, including the wires that comprise the bus 1902. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to the processor 1904 for execution. For example, the instructions may initially be carried on a magnetic disk of a remote computer. The remote computer can load the instructions into its dynamic memory and send the instructions over a telephone line using a modem. A modem local to the computer system 1900 can receive the data on the telephone line and use an infrared transmitter to convert the data to an infrared signal. An infrared detector coupled to the bus 1902 can receive the data carried in the infrared signal and place the data on the bus 1902. The bus 1902 carries the data to the main memory 1906, from which the processor 1904 retrieves and executes the instructions. The instructions received by the main memory 1906 may optionally be stored on the storage device 1910 either before or after execution by the processor 1904.

The computer system 1900 also includes a communication interface 1918 coupled to the bus 1902. The communication interface 1918 provides a two-way data communication coupling to a network link 1920 that is connected to a local network 1922. For example, the communication interface 1918 may be an integrated services digital network (ISDN) card or a modem to provide a data communication connection to a corresponding type of telephone line. As another example, the communication interface 1918 may be a local area network (LAN) card to provide a data communication connection to a compatible LAN. Wireless links may also be implemented. In any such implementation, the communication interface 1918 sends and receives electrical, electromagnetic or optical signals that carry data streams representing various types of information.

The network link 1920 typically provides data communication through one or more networks to other devices. For example, the network link 1920 may provide a connection through local network 1922 to a host computer 1924 or to equipment 1926 such as a radiation beam source or a switch operatively coupled to a radiation beam source. The data streams transported over the network link 1920 can comprise electrical, electromagnetic or optical signals. The signals through the various networks and the signals on the network link 1920 and through the communication interface 1918, which carry data to and from the computer system 1900, are exemplary forms of carrier waves transporting the information. The computer system 1900 can send messages and receive data, including program code, through the network(s), the network link 1920, and the communication interface 1918.

Although particular embodiments have been shown and described, it will be understood that it is not intended to limit the claimed inventions, and it will be obvious to those skilled in the art that various changes and modifications may be made without departing from the spirit and scope of the claimed inventions. The specification and drawings are, accordingly, to be regarded in an illustrative rather than restrictive sense. The claimed inventions are intended to cover alternatives, modifications, and equivalents.

What is claimed:

1. A medical method, comprising:
obtaining marker positions at a plurality of respective time points;
determining a first subset of the marker positions that belongs to a first phase bin;
using the marker positions in the first subset to determine a first variance information, wherein the first variance information is determined using a processor; and
storing the first variance information in a non-transitory medium.

2. The method of claim 1, wherein the obtained marker positions comprises positions of a marker inside a patient.

3. The method of claim 1, wherein the obtained marker positions comprises positions of a marker outside a patient.

4. The method of claim 1, further comprising:
determining a second subset of the marker positions that belongs to a second phase bin; and
using the marker positions in the second subset to determine a second variance information.

5. The method of claim 1, wherein the first variance information comprises a probability distribution of the marker positions in the first set.

6. The method of claim 5, further comprising transforming the probability distribution in a marker space to a tissue space.

7. The method of claim 6, wherein the transformed probability distribution in the tissue space represents uncertainty of a position of a tissue due to motion, and the method further comprises displaying a graphic representing the uncertainty of the position of the tissue.

8. The method of claim 7, wherein the graphic comprises a plurality of lines or a color map surrounding an image or a contour of the tissue.

9. The method of claim 1, wherein the first variance information comprises a probability distribution of the marker positions in the first set, and the method further comprises displaying a graphic in a screen that is associated with the probability distribution of the marker positions in the first set.

10. The method of claim 9, wherein the graphic comprises a plurality of lines or a color map surrounding an image or a contour of a tissue.

11. The method of claim 9, wherein the graphic comprises a plurality of lines or a color map surrounding an indicator of a marker.

12. The method of claim 9, wherein the graphic represents different probability values that are above a set threshold.

13. The method of claim 1, wherein the first variance information comprises a parameter representing a number of one or more marker positions in the first set that are at a same location or within a same spatial area.

14. The method of claim 1, wherein the first variance information is determined by:
determining whether one of the marker positions is at a same location or within a same spatial area as that of another one of the marker positions; and
incrementing a count number associated with the location or the spatial area if the one of the marker positions is at the same location or within the same spatial area as that of the other one of the marker positions.

15. The method of claim 1, further comprising:
obtaining image data; and
associating the image data with the first variance information.

16. The method of claim 15, wherein the image data comprises CT image data, x-ray image data, PET image data, MR image data, SPECT image data, PET-CT image data, or ultrasound image data.

17. The method of claim 16, wherein the marker positions are obtained during a treatment procedure.

18. The method of claim 17, wherein the treatment procedure comprises a radiation treatment procedure.

19. The method of claim 16, wherein the marker positions are obtained during a 4D imaging procedure.

20. The method of claim 16, wherein the marker positions are obtained during a data collection procedure that does not involve imaging or treatment of a patient.

21. The method of claim 1, further comprising using the first variance information to determine a first parameter in a treatment plan.

22. The method of claim 21, wherein the treatment plan comprises a radiation treatment plan.

23. The method of claim 21, wherein the first parameter comprises a first treatment margin that is determined during a treatment session.

24. The method of claim 23, further comprising:
determining a second subset of the marker positions that belongs to a second phase bin;
using the marker positions in the second subset to determine a second variance information; and
using the second variance information to determine a second treatment margin;
wherein the first margin corresponds to a first phase of a physiological, and the second margin corresponds to a second phase of the physiological.

25. The method of claim 21, wherein the first parameter comprises a gating window for activating a radiation beam.

26. An apparatus, comprising:
a processing unit configured for:
obtaining marker positions at a plurality of respective time points,
determining a first subset of the marker positions that belongs to a first phase bin, and
using the marker positions in the first subset to determine a first variance information; and
a non-transitory medium for storing the first variance information.

27. The apparatus of claim 26, wherein the processing unit is further configured for:
determining a second subset of the marker positions that belongs to a second phase bin; and
using the marker positions in the second subset to determine a second variance information.

28. The apparatus of claim 26, wherein the first variance information comprises a probability distribution of the marker positions in the first set.

29. The apparatus of claim 28, wherein the processing unit is further configured for transforming the probability distribution in a marker space to a tissue space.

30. The apparatus of claim 29, wherein the transformed probability distribution in the tissue space represents uncertainty of a position of a tissue due to motion, and the processing unit is further configured to output a graphic for display in a screen, the graphic representing the uncertainty of the position of the tissue.

31. The apparatus of claim 30, wherein the graphic comprises a plurality of lines or a color map surrounding an image or a contour of the tissue.

32. The apparatus of claim 26, wherein the first variance information comprises a probability distribution of the marker positions in the first set, and the processing unit is further configured to output a graphic for display in a screen, the graphic associated with the probability distribution of the marker positions in the first set.

33. The apparatus of claim 32, wherein the graphic comprises a plurality of lines or a color map surrounding an image or a contour of a tissue.

34. The apparatus of claim 32, wherein the graphic comprises a plurality of lines or a color map surrounding an indicator of a marker.

35. The apparatus of claim 32, wherein the graphic represents different probability values that are above a set threshold.

36. The apparatus of claim 26, wherein the first variance information comprises a parameter representing a number of one or more marker positions in the first set that are at a same location or within a same spatial area.

37. The apparatus of claim 26, wherein the processing unit is configured to determine the first variance information by:
determining whether one of the marker positions is at a same location or within a same spatial area as that of another one of the marker positions; and
incrementing a count number associated with the location or the spatial area if the one of the marker positions is at the same location or within the same spatial area as that of the other one of the marker positions.

38. The apparatus of claim 26, wherein the processing unit is further configured for:
obtaining image data; and
associating the image data with the first variance information.

39. The apparatus of claim 38, wherein the image data comprises CT image data, x-ray image data, PET image data, MR image data, SPECT image data, PET-CT image data, or ultrasound image data.

40. The apparatus of claim 26, wherein the processing unit is further configured to use the first variance information to determine a first parameter in a treatment plan.

41. The apparatus of claim 40, wherein the treatment plan comprises a radiation treatment plan.

42. The apparatus of claim 40, wherein the first parameter comprises a first treatment margin, and the processing unit is configured to determine the first treatment margin during a treatment session.

43. The apparatus of claim 42, wherein the processing unit is further configured for:
determining a second subset of the marker positions that belongs to a second phase bin;
using the marker positions in the second subset to determine a second variance information; and
using the second variance information to determine a second treatment margin;
wherein the first margin corresponds to a first phase of a physiological, and the second margin corresponds to a second phase of the physiological.

44. The apparatus of claim 40, wherein the first parameter comprises a gating window for activating a radiation beam.

45. A computer product having a non-transitory medium storing a set of instructions, and execution of which causes a method to be performed, the method comprising:
obtaining marker positions at a plurality of respective time points;
determining a first subset of the marker positions that belongs to a first phase bin;
using the marker positions in the first subset to determine a first variance information, wherein the first variance information is determined using a processor; and
storing the first variance information.

46. The computer product of claim 45, wherein the method further comprises:
determining a second subset of the marker positions that belongs to a second phase bin; and
using the marker positions in the second subset to determine a second variance information.

47. The computer product of claim 45, wherein the first variance information comprises a probability distribution of the marker positions in the first set.

48. The computer product of claim 47, wherein the method further comprises transforming the probability distribution in a marker space to a tissue space.

49. The computer product of claim 48, wherein the transformed probability distribution in the tissue space represents uncertainty of a position of a tissue due to motion, and the method further comprises outputting a graphic for display in a screen, the graphic representing the uncertainty of the position of the tissue.

50. The computer product of claim 49, wherein the graphic comprises a plurality of lines or a color map surrounding an image or a contour of the tissue.

51. The computer product of claim 45, wherein the first variance information comprises a probability distribution of the marker positions in the first set, and the method further comprises outputting a graphic for display in a screen, the graphic associated with the probability distribution of the marker positions in the first set.

52. The computer product of claim 51, wherein the graphic comprises a plurality of lines or a color map surrounding an image or a contour of a tissue.

53. The computer product of claim 51, wherein the graphic comprises a plurality of lines or a color map surrounding an indicator of a marker.

54. The computer product of claim 51, wherein the graphic represents different probability values that are above a set threshold.

55. The computer product of claim 45, wherein the first variance information comprises a parameter representing a number of one or more marker positions in the first set that are at a same location or within a same spatial area.

56. The computer product of claim 45, wherein the first variance information is determined by:
determining whether one of the marker positions is at a same location or within a same spatial area as that of another one of the marker positions; and
incrementing a count number associated with the location or the spatial area if the one of the marker positions is at the same location or within the same spatial area as that of the other one of the marker positions.

57. The computer product of claim 45, wherein the method further comprises:
obtaining image data; and
associating the image data with the first variance information.

58. The computer product of claim 57, wherein the image data comprises CT image data, x-ray image data, PET image data, MR image data, SPECT image data, PET-CT image data, or ultrasound image data.

59. The computer product of claim 45, wherein the method further comprises using the first variance information to determine a first parameter in a treatment plan.

60. The computer product of claim 59, wherein the first parameter comprises a first treatment margin that is determined during a treatment session.

61. The computer product of claim 59, wherein the first parameter comprises a gating window for activating a radiation beam.
